SEQUENCE LISTING (3 sequences)

SEQ ID NO: 1 - DNA, 25 bp, Artificial Sequence
  Guide RNA (gRNA) sequence CR1 for targeting      NDST1 in renal carcinoma cells.
caccgatgacgcacctgtccaacta

SEQ ID NO: 2 - DNA, 25 bp, Artificial Sequence
  Guide RNA (gRNA) sequence CR2 for targeting      NDST1 in renal carcinoma cells.
caccgtgcactcctggacgaacctc

SEQ ID NO: 3 - DNA, 25 bp, Artificial Sequence
  Guide RNA (gRNA) sequence CR2 for targeting      HAS1 in renal carcinoma cells.
caccgccgcgaggtcttcgctgacg

(19) United States
(12) Patent Application Publication
(10) Pub. No.: US 2019/0144867 A1
(43) Pub. Date: May 16, 2019

(54) METHOD FOR MITIGATING METASTASIS

(71) Applicants: RESEARCH FOUNDATION OF THE CITY UNIVERSITY OF NEW YORK, New York, NY (US); MEMORIAL SLOAN KETTERING CANCER CENTER, New York, NY (US); THE GENERAL HOSPITAL CORPORATION D/B/A MASSACHUSETTS GENERAL HOSPITAL, Boston, MA (US)

(72) Inventors: John M. TARBELL, New York, NY (US); Lance L. MUNN, Boston, MA (US); Henry QAZI, New York, NY (US); Zhongdong SHI, New York, NY (US)

(73) Assignees: Research Foundation of the City University of New York, New York, NY (US); Memorial Sloan Kettering Cancer Center, New York, NY (US); The General Hospital Corporation D/B/A Massachusetts General Hospital, Boston, MA (US)

(21) Appl. No.: 15/760,663

(22) PCT Filed: Sep. 19, 2016
(86) PCT No.: PCT/US2016/052446

§ 371 Date: Mar. 16, 2018

Related U.S. Application Data
(60) Provisional application No. 62/219,882, filed on Sep. 17, 2015.

(51) Int. Cl. C12N 15/113; A61P 35/04; C12N 9/22; C12N 15/11; A61K 38/51; A61K 31/519; A61K 31/336

(52) U.S. Cl. CPC C12N 15/1137; A61P 35/04; C12N 9/22; C12N 15/11; C12Y 402/02008; C12N 2800/80; A61K 31/519; A61K 31/336; C12N 2310/122; C12N 2310/531; C12N 2310/20; A61K 38/51

(57) ABSTRACT

A method for inhibiting metastasis of cancer cells is provided. Inhibitors of heparan sulfate (HS) or hyaluronic acid (HA) are applied to a tumor or a surgical location after removal of the bulk of the tumor. The inhibitors enzymatically cleave surface HS or HA; genetically modify cancer cells to decrease HS or HA production or interfere with the signaling pathway between HS or HA and a MMP or syndecan.

### CROSS-REFERENCE TO RELATED APPLICATIONS

This application claims priority to and is a non-provisional of U.S. Patent Application 62/219,882 (filed Sep. 17, 2015), the entirety of which is incorporated herein by reference.

### STATEMENT OF FEDERALLY SPONSORED RESEARCH OR DEVELOPMENT

This invention was made with Government support under grant number RO1 HL094889 awarded by the National Institute of Health (NIH). The government has certain rights in the invention.

### REFERENCE TO A SEQUENCE LISTING

This application refers to a “Sequence Listing” listed below, which is provided as an electronic document entitled “17588_ST25.txt” (2 kilobytes created on Sep. 19, 2016) which is incorporated herein by reference in its entirety.

### BACKGROUND OF THE INVENTION

The subject matter disclosed herein relates to methods for inhibiting metastasis of cancer cells. Certain cancer cells are more prone than others to undergo metastasis. While attempts have been made to develop therapies that inhibit or prevent metastasis, these attempts have not been entirely successful or are not applicable in all situations. Improved methods for inhibiting metastasis are therefore desired.

The discussion above is merely provided for general background information and is not intended to be used as an aid in determining the scope of the claimed subject matter.

### BRIEF DESCRIPTION OF THE INVENTION

A method for inhibiting metastasis of cancer cells is provided. Inhibitors of heparan sulfate (HS) or hyaluronic acid (HA) are applied to a tumor or to a surgical location after removal of the bulk of the tumor. The inhibitors genetically modify residual cancer cells to decrease HS or HA production; enzymatically cleave surface HS or HA; or interfere with the signaling pathway between HS or HA and a MMP or syndecan.

In a first embodiment, a method for inhibiting metastasis of cancer cells is provided. The method comprising treating an in vivo tumor with a glycosaminoglycan (GAG) inhibitor, the glycosaminoglycan (GAG) inhibitor selected from a group consisting of a heparan sulfate (HS) inhibitor and a hyaluronic acid (HA) wherein the glycosaminoglycan (GAG) inhibitor causes a decrease in glycosaminoglycan concentration on a surface of cells in the in vivo tumor relative to untreated cells.

In a second embodiment, a method for inhibiting metastasis of cancer cells is provided. The method comprising sequential steps of surgically removing at least a portion of a tumor from an in vivo surgical location of a patient; and treating the in vivo surgical location with a glycosaminoglycan (GAG) inhibitor, the glycosaminoglycan (GAG) inhibitor selected from the group consisting of a heparan sulfate (HS) inhibitor and a hyaluronic acid (HA) wherein the glycosaminoglycan (GAG) inhibitor causes a decrease in glycosaminoglycan concentration on a surface of cells in the in vivo surgical location relative to untreated cells, thereby inhibiting metastasis of any residual cancer cells in the in vivo surgical location.

In a third embodiment, a method for inhibiting metastasis of cancer cells is provided. The method comprising steps of surgically removing at least a portion of a renal carcinoma tumor from an in vivo surgical location of a patient; and treating the in vivo surgical location with a heparan sulfate (HS) inhibitor that causes a decrease in glycosaminoglycan concentration on a surface of cells in the in vivo surgical location relative to untreated cells, thereby inhibiting metastasis of any residual renal carcinoma tumor cells in the in vivo surgical location; wherein the heparan sulfate (HS) inhibitor is a shRNA transfected into a N-deacetylase/N-sulfotransferase1 (NDST1) gene of the cells in the in vivo surgical location.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1A is graph depicting NDST1 gene expression of two transfected control cell lines (CtGFP, CtORG) and four cell lines with NDST1 knocked down by short-hairpin RNA methods (shGFP-14, shGFP-17, shORG-8, shORG-10);

FIG. 1B is a graph showing proliferation rates of the cell lines of FIG. 1A;

FIG. 2A is graph depicting relative migration of the cell lines in response to flow in the Boyden chamber;

FIG. 2B is a graph depicting baseline (no flow) migration rates of the cell lines;

FIG. 3 shows the results of a T7EI assay showing successful incorporation of mutations in NDST1 and HAS1 genes by CRISPR plasmid transfection;

FIG. 4 is a graph depicting migration response of high metastatic renal carcinoma cells after exposure to interstitial flow following GAG enzymatic cleavage; and

FIG. 5 is a graph depicting normalized migration response of metastatic renal kidney carcinoma cells to interstitial flow after in vitro treatment with MAPK signaling inhibitors.

### DETAILED DESCRIPTION OF THE INVENTION

Disclosed in this specification are methods for inhibiting the production of, inhibiting the function of or removing heparan sulfate (HS) and/or hyaluronic acid (HA) glycosaminoglycans (GAGs) from a surface of cancer cells to prevent the cancer cells from migrating (metastasizing) from a primary tumor to local or distant secondary sites. Three approaches are described including (1) reducing metastasis using genetic methodologies to inhibit GAGs production (2) reducing metastasis by depleting the GAGs on the cell surface using enzymes and (3) reducing metastasis by blockading GAGs signaling using agents that interfere with the association of the GAGs with intracellular signaling mechanisms that support metastasis. Each approach is discussed in further detail elsewhere in this specification. Loss of cell surface HS and/or HA significantly reduces the ability of cells to invade locally or metastasize to distant sites.

Heparan sulfate and hyaluronic acid are the dominant glycosaminoglycans on most cancer cell surfaces. See J. Cell. Mol. Med. Vol 15, No. 5, 2011 pp. 1013-1031. HS is a sulfated GAG, and forms proteoglycans when bound covalently to a protein core. The proteoglycan core proteins may be incorporated into the cell membrane by a GPI anchor (e.g. glypicans) or a transmembrane domain that links to the cytoskeleton (e.g. syndecans) while the GAG chains extend into the extracellular space and sense the mechanical and chemical environment of the cancer cell. This specification describes proteoglycans bound to cancer cell surfaces such as syndecans and glypicans, not those proteoglycans that may be present in the extracellular matrix but not bound to the cell surface (e.g., perlecan). HA is a non-sulfated GAG of extended length that binds to cell surface receptors (e.g., CD44). Again, this disclosure is referring to the cell-bound HA not the free HA in the extracellular matrix.

There have been some studies (e.g. PLoS ONE, January 2011; Vol. 6, Issue 1, to Zhong-Dong Shi et al.) concerning heparin sulfate proteoglycans and cell motility but these studies only utilize in vitro models and deal with vascular smooth muscle cells only.

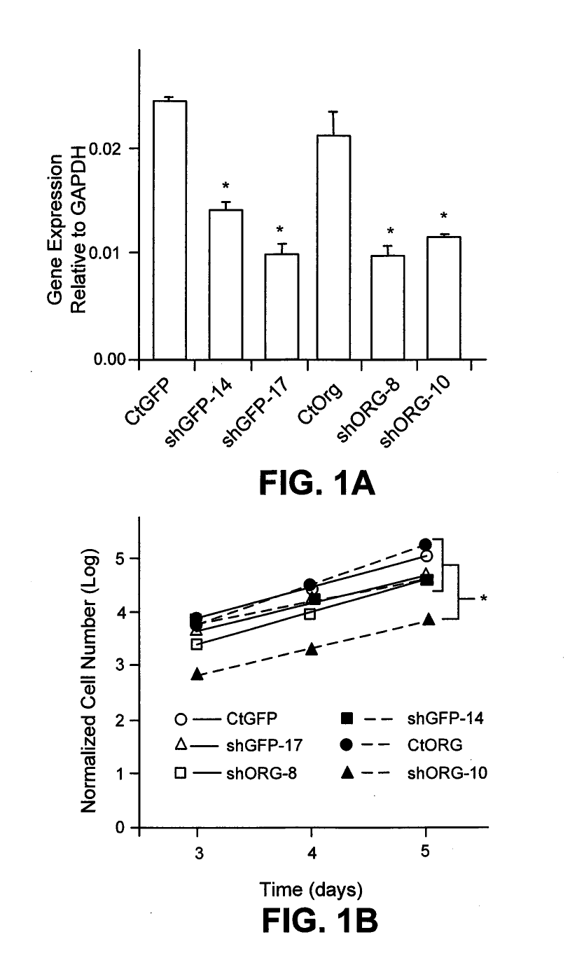

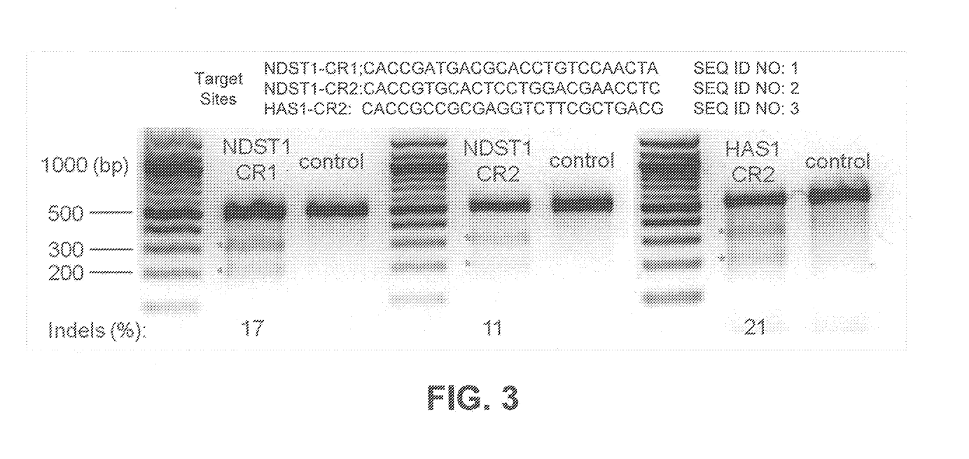

In a first embodiment, a method is provided that uses genetic methodologies (e.g. shRNA, CRISPR-Cas9) to reduce key enzymes in the synthetic pathway leading to HS production. A specific example is N-deacetylase/N-sulfotransferase1 (NDST1) shRNA transfection, which reduces expression of HS in cancer cells (FIG. 1A) and suppresses renal cell carcinoma metastasis in a mouse model. For HA, a key synthetic enzyme, hyaluronan synthase 1 (HAS1), can be reduced by CRISPR-Cas9 methods (FIG. 3) and shRNA

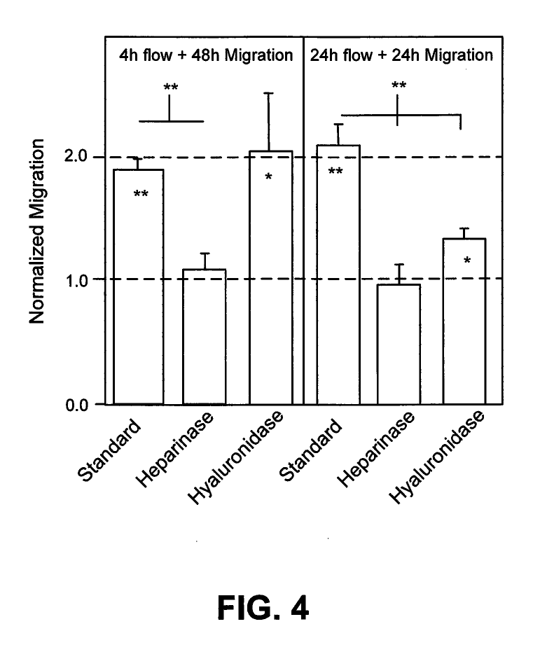

In a second embodiment, a method is provided that uses enzymes that specifically cleave HS or HA from the cancer cell surface. In vitro studies have shown that when the enzymes heparinase III or hyaluronidase are applied to renal carcinoma cells to degrade the cell surface GAGs, the migration of these cells in response to interstitial flow in a 3 dimensional gel model is greatly suppressed. See FIG. 4. Also see Integr Biol (Camb) 2013; 5(11): 1334-43.

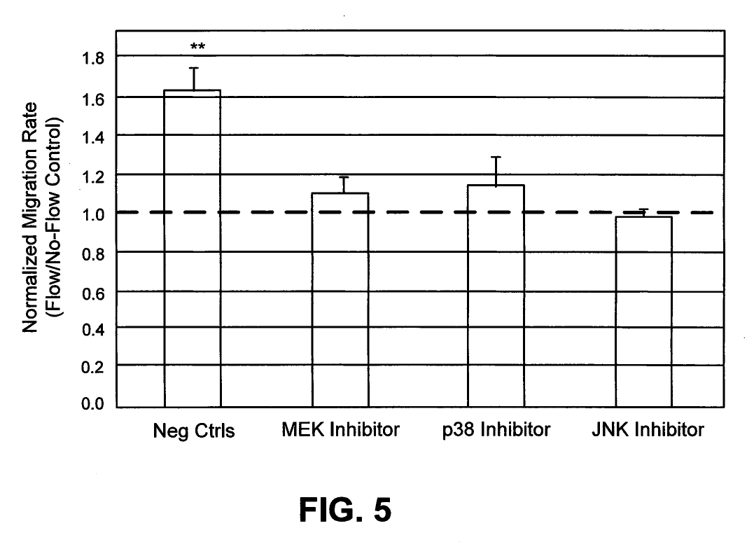

In a third embodiment, a method is provided that uses inhibitors to blockade GAGs signaling to downstream effectors of migration (MMPs, integrins). Examples include inhibitors of the intracellular mitogen-activated protein kinase (MAPK) pathways that have been shown to mediate cell migration in vascular smooth muscle cells and cancer cells in vitro and are believed to be downstream effectors of surface sensing by HS and HA. See FIG. 5. In vitro studies of cancer cell migration have shown that MEK, p38 and JNK inhibitors suppress migration significantly.

Each of these embodiments may be used in conjunction with traditional therapies including, systemic delivery of agents, local delivery of agents through nanoparticle technology, as well after surgical removal of tumors. During surgical removal the tumor site may be treated with one or more of the disclosed methods to suppress metastasis of any cancer cells that may evade surgical removal.

Section 1: Genetic Methodologies to Reduce HS Production

Highly metastatic kidney carcinoma cells (SN12L1) were genetically modified using short hair-pin RNA (shRNA) to knockdown the NDST1 gene, and then injected into the kidney capsule of SCID mice. The metastasis to distant organs was quantified after sectioning and staining for metastatic kidney carcinoma cells. A typical result for the lung, which was the most common metastatic site, showed metastasis was greatly suppressed in the NDST1 knockdown cells. For example, fluorescence imaging of lung tissue samples collected from mice implanted with either control SN12L1 cells or NDST1-knockdown cells in the kidney shows significant metastasis suppression. The imaging data was quantified for all organs and the overall results are displayed in Table 1. The dramatic inhibition of metastasis is apparent in this table, and the overall suppression of metastasis of the NDST1 knockdown cells was 95%.

HS production was inhibited in the knockdown clones. Lentiviral vector transfer of NDST1 snRNA into the SN12L1 metastatic cells yielded cell lines with a 40-60% reduction in NDST1 mRNA expression (FIG. 1A). Cell viability stabilized after 2 days of growth (p<0.005), and all cell lines exhibited log phase growth (FIG. 1B, day 3-5). Linear regression analysis for these time points showed linear correlations (r²>0.99) for each cell line. Only shORG-10 differed significantly from other cell lines (p<0.001), and was not used for further studies. There was also an observable reduction in HS expression in the NDST1 knockdown lines, as expected. Analysis of the confocal volumes and transverse plane images revealed that HS staining was restricted to cell surfaces, with no intracellular staining. Interestingly, differences were found when analyzing HS expression in 2D cultures and 3D gels: NDST1-silenced cells showed a 25-35% reduction in HS expression in 2D culture but a 42-75% reduction when cultured in 3D gels.

Flow-Induced Migration is Suppressed by Knockdown of HS

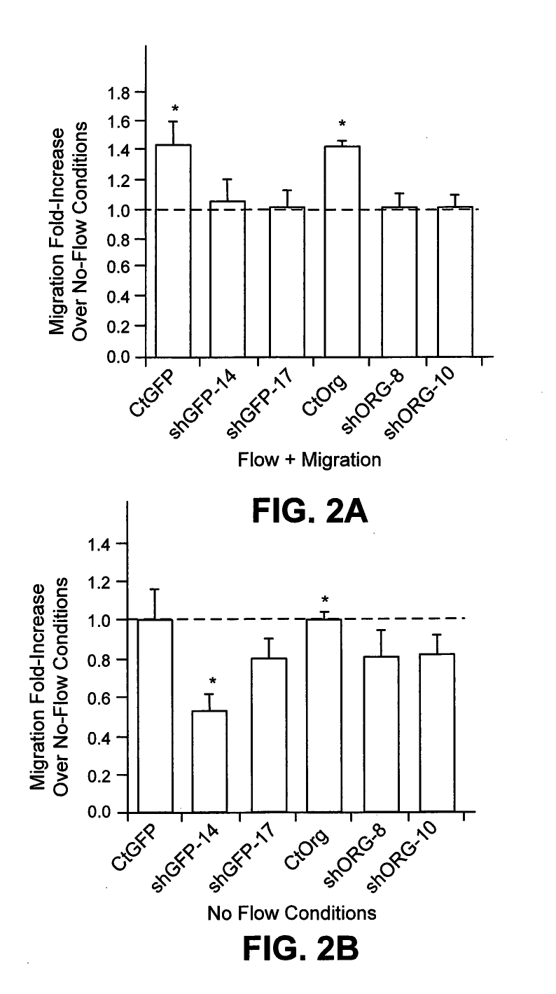

FIG. 2A and FIG. 2B depict in vitro migration and invasion assays in a modified Boyden chamber. FIG. 2A shows migration in response to flow in the Boyden chamber model. The control CtGFP and CtORG lines showed greater than 40% flow-mediated upregulation in migration (* p<0.05, n=4); NDST1 knockdown cell lines showed no upregulation of migration in response to flow (p>0.5, n=4-6). FIG. 2B provides baseline (no flow) migration rates. Note that shGFP data were normalized by CtORG values and shORG data were normalized by CtGFP values. Baseline migration was significantly lower for the shGFP-14 cell line (* p<0.05, n=4); other cell lines were not affected significantly (p>0.05, n=4-6). CtGFP and shORG-8 lines were selected as the representative pairs for the remaining invasion models in mice.

Knockdown of HSPG Expression Suppresses Distant Colonization

With the phenotypes of the cells established in the in vitro analyses, the ability of the cells to invade and metastasize were tested in a SCID mouse model of primary renal carcinoma. The modified cell lines (CtGFP and shORG-8) were injected into the kidney capsule, and after four weeks of growth, the primary tumors were removed (via nephrectomy) and the secondary colonies were allowed to grow until tumor burden was evident. At this point, the mice were sacrificed and tissues were harvested for analysis.

Table 1 quantifies HS knockdown induced reduction in invasive tumor colonies per tissue sample for both the single clone and the combined clones models*.

TABLE 1

| | Single Clone Model | | | | | |
|---|---|---|---|---|---|---|
| | | Knock- | | Combined Clones Model | | |
| | Control | down | Δ% | Control | Knockdown | Δ% |
| Kidney | 1.8 | 0.3 | −86 | 0.8† | 0.0 | −100† |
| Liver | 1.7 | 0.1 | −97 | 2.1 | 0.1 | −94 |
| Lungs | 14.6 | 0.5 | −97 | 7.2 | 0.1 | −98 |
| Abdomen | 3.3 | 0.3 | −92 | 2.6 | 0.0 | −100 |
| Spleen | 1.8 | 0.0 | −100 | 0.4† | 0.0 | −100† |

*p < 0.005 for each comparision between the number of invasive tumor colonies formed by either the control (CtGFP) or knockdown (shORG-8) cell lines within each tissue type**
†0.005 < p < 0.05 for the samples in the combined clones models were there were fewer observed cases of invasive colonies formed by the control (GtGFP) cell line
**n = 165 tissue samples for the single clone control case, n = 66 tissue samples for the single clone knockdown case and n = 50 for the paired cases in the combined clones model

The presence of multiple invading and metastasizing GFP-expressing CtGFP (control) cells was evident in all tissues analyzed. This was true in both the single and mixed clone models. In tumors containing only the ORG-expressing shORG-8 (NDST1 knockdown) cells, there were large primary tumors (detected at the nephrectomy), but very few metastatic colonies in any of the tissue samples. In contrast, control (CtGFP) tumors resulted in multiple metastatic lesions in the liver, contralateral kidney, lungs, abdomen and spleen. Overall, there were 86-100% fewer invasive tumor colonies in the shORG group compared with the CtGFP group (Table 1). When the primary tumor contained a mixture of CtGFP and shORG cells, nearly all of the metastatic colonies consisted of CtGFP cells, although a few small shORG colonies were detected outside the primary tumor mass. The lungs were the most infiltrated distant site, with greatest number of fluorescent tumor colonies per tissue sample (Table 1). Consequently, the contrast was greatest for this tissue, with knockdown of NDST1 leading to a 97% decrease in lung colonies compared to the control tumors.

Knockdown of HSPG Suppresses Invasive Phenotype

In addition to this difference in distant colonization, there were important differences in the morphologies of the primary tumor boundaries. Examining H&E sections, the shORG-8 tumors were found to be large in size (low metastasis from the primary tumor) but had well-defined boundaries with the surrounding normal tissue. In contrast, the CtGFP colonies had poorly defined margins, and appeared to invade the surrounding tissue more aggressively (Table 2).

TABLE 2

| | Control | Knockdown | Δ % |
|---|---|---|---|
| % Tumor masses | 92 | 16 | −82 |
| that are invasive | | | |
| % Tumor colony | 32 | 80 | +149 |
| sizes >1 mm | | | |

*p < 0.005 for each comparison between the invasive rates and colony sizes for the control (CtGFP) or knockdown (shORG-8) cell lines **
** Tumor cells at the tumor/normal tissue interface were evaluated for both the control (CtGFP; n = 774 and knockdown (shORG-8; n = 67) cell lines. Secondary tumor colonies formed by both the control (CtGFP; n = 818) and knockdown (shORG-8; n = 75) cell lines were evaluated for size.

CtGFP cells frequently invaded the kidneys, spleen, and other abdominal organs. In contrast, minimal invasion of the abdominal cavity was observed by NDST1 knockdown tumors. Because knockdown cells did not readily invade surrounding normal tissue, the shORG-8 tumors grew locally and formed larger isolated tumors compared to control tumors (Table 2).

Discussion

Interstitial fluid flow near the tumor margin is associated with poor survival in patients with cervical carcinoma. In vivo tumor interstitial flow rates, measured by either implanted devices or dynamic contrast-enhanced MRI, are significantly elevated to various levels depending on the type of tumor. Many in vitro models encompassing several cell lines have demonstrated that interstitial flow and fluid shear stress act on tumor cells to affect focal adhesions, integrin expression, stromal cell recruitment, MMP activation and expression, and invasion rates. The disclosed methods recognize that HS on the cancer cell surface glycocalyx mediates migration and metastasis, possibly by the potentiation of interstitial flow signals.

Knocking down HS expression did not significantly affect baseline migration of the shORG-8 cells used in the animal experiments, but inhibited flow-induced invasion, suggesting that the glycocalyx can serve as a mechanosensor for highly metastatic kidney carcinoma cells.

Tumor cell invasive and metastatic potential can be assessed by observing and quantifying distant organ sites that have been seeded by tumor cells originating from a single primary tumor. Fluorescence imaging of GFP-expressing and ORG-expressing tumor cells allowed us to quantify metastatic colonies at different organ sites (Table 1). Control cells (CtGFP) formed highly metastatic tumors, while HS knockdown cells (shORG-8) formed tumors with lower metastatic potential (Table 1). For the paired comparison of both cell types in the mixed model, there was very little evidence that NDST1 knockdown cells metastasized or invaded to form metastatic tumors (Table 1).

Interestingly, tunneling of CtGFP cells into the tissue did not create pathways for subsequent invasion of the knockdown cells, as has been suggested in in vitro studies. This suggests there is a fundamental deficiency in the migration machinery rather than simply an inability to degrade the extracellular matrix introduced by the lack of HS. However, there may also be decreased matrix protease activity in the knockdown cells, as they consistently formed well circumscribed boundaries with the normal tissue, apparently unable to degrade the matrix at the interface (Table 2). On the other hand, control (CtGFP) tumors formed multiple leading edges and aggressively invaded normal tissue, whereas NDST1 knockdown cells remained confined, did not invade organ capsules, and formed tumors with well circumscribed boundaries (Table 2). Changes in invasive rates of connective and muscular tissue were a prime example of how downregulation of HSPG could diminish the invasive phenotype of highly metastatic cells. Because control cells were aggressively invasive, less than a third of the distant tumor masses grew to greater than 1 mm in size before disseminating (Table 2). In contrast, cells with downregulation of HSPG formed tumors that were locally confined to the abdominal cavity and grew to larger sizes without invading (Table 2). Although the in vitro results suggest that these differences in metastasis and invasion may be the result of altered flow mechanosensing in the knockdown cells, interstitial flow and invasion could not be controlled or measured in the animal studies. This is due to the lack of current methodologies for monitoring or controlling interstitial flow non-invasively over the course of the 3 month experiments.

There have been seemingly conflicting reports in the literature about the role of HS in tumor growth and metastasis. HSPGs play a complex role that could inhibit or enhance tumor cell invasion and metastasis. For example, HS in the extracellular matrix enhances barrier properties that helps to limit cell migration, and binds growth factor and signaling molecules thus shielding cancer cells and suppressing growth and metastasis. On the other hand, HSPGs linked to the cell surface have been described as essential for tumor growth, enhancing polyamine presentation to the cell thereby supporting cell proliferation and B cell-mediated immune reaction. Cell-surface receptor functions can also be enhanced by upregulation of the glycocalyx that could lead to an increase in cell survival, growth, and spread.

Currently, limited therapeutics exist that target degradation of the glycocalyx. To remove HS, naphthalene methanol-D-xyloside (NX), a drug that blocks the assembly of HS side chains of proteoglycans has been described. To deplete hyaluronans, pegylated human recombinant hyaluronidase (PEGPH20) has been used in preclinical and clinical studies to enhance perfusion and drug delivery. Unfortunately, systemic delivery of such drugs can have serious side effects by depleting the endothelial cell glycocalyx, which provides a barrier to vascular leakage and inflammatory cell adhesion and sustains flow-induced release of nitric oxide. Though challenges remain for delivery of shRNA to knockdown specific glycocalyx components on cancer cells, a number of methods, including nanoparticle delivery, are being developed for such purposes.

A number of gene editing platforms including CRISPR/Cas9 are suitable for the delivery of genetic modifications to tumor cells. See Annu. Rev. Chem. Biomol Eng. 2016. 7:637-62. It is possible to inject CRISPRs in adeno-associated viruses (AAVs) locally in solid tumors. These methods, and others, may be used for mutating NDST1 and HAS1 to reduce HS and HA on cancer cell surfaces.

CRISPRs have been identified for both NDST1 and HAS1 thus demonstrating the technology for these enzymes. Three guide RNA (gRNA) sequences from GeCKOv2 Human Library were randomly chosen (see FIG. 3, SEQ ID NO: 1; SEQ ID NO: 2 and SEQ ID NO: 3) targeting NDST1 and HAS1 genes. The gRNA sequences were cloned into the pX330 vector containing Cas9. The plasmids were transfected into the SN12L1 cells using Lipofectamine 3000 (invitorgen). Genomic DNA was isolated for T7 endonuclease I (T7EI) assay to assess the targeting efficiency. The T7EI assay showed that one could efficiently generate about 20% indel (insertion and deletion) mutations in NDST1 and HAS1 genes by CRISPR plasmid transfection. With this high efficiency, knockout clonal lines can be established by sub-cloning these cells.

Section 2: Inhibiting Metastasis by Depleting HS or HA on Cancer Cell Surface Using an Enzyme

In a second embodiment, enzymes are applied to an in vivo tumor location to suppress metastasis. The treatment may be applied systemically, by local injection, for example, immediately after removal of the majority of the tumor by surgery. The treatment is applied to the surgical site to inhibit metastasis of any cancer cells that may have evaded surgical removal. Examples of suitable enzymes include heparinase III and hyaluronidase. In one embodiment such examples are applied to renal carcinoma cells to degrade the cell surface GAGs. In response the migration of these cells is greatly suppressed.

FIG. 4 depicts migration response of highly metastatic renal carcinoma cells (SN12L1) after 4h and 24h exposure to interstitial flow following GAG cleavage with either heparinase or hyaluronidase. Migration rates are normalized by no-flow controls. Hyaluronidase and heparinase significantly inhibit the flow-enhanced invasive potential of the SN12L1 cells.

Section 3:Inhibiting Metastasis by Blockaiding HS Signaling Using Agents that Interfere with Association of GAGs with Grow Factor Receptors.

In a third embodiment, HS and/or HA signals to MMPs and integrins that enhance migration are blocked by treating with an inhibitor. Examples include inhibitors of the intracellular mitogen-activated protein kinase (MAPK) pathways that have been shown to mediate cell migration in vascular smooth muscle cells and cancer cells in vitro and are believed to be downstream effectors of surface sensing by HS and HA. In vitro studies of cancer cell migration have shown that MEK, p38 and JNK inhibitors suppress migration significantly. Examples of suitable inhibitors that have been used in human subjects include GNE-470, trametinib, and fucoxanthin.

FIG. 5 depicts normalized migration response of metastatic renal kidney carcinoma cells (SN12L1) to interstitial flow with MAPK signaling inhibitors in vitro. Negative controls correspond to cases without pathway inhibition. MEK, p38, and JNK were specifically inhibited. The inhibitors were: U0126—MEK1 and MEK2 inhibitor, SB 203580—P38MAPK inhibitor, JNK Inhibitor II—JNK 1, JNK 2, and JNK 3 inhibitors. The results are presented as flow normalized to no-flow controls. Blockers of the MAPK signaling pathways inhibited flow-induced enhancement of migration in all cases.

Materials and Methods

Establishment and Characterization of NDST1 Knockdown Lines

SN12L1, human metastatic renal carcinoma cells (courtesy of Dr. Isaiah Fidler, MD Anderson Cancer Center) were cultured following MD Anderson protocols. To knock down NDST1, SN12L1 cells were infected with NDST1 shRNA lentiviral particles containing 3 target-specific constructs (Santa Cruz Biotechnology, sc-40761-V); cells infected with shRNA lentiviral particles containing GFP (sc-108084) served as knockdown control cells. Puromycin selection was performed to remove uninfected cells and to establish stable cell lines. To label the NDST1 knockdown cells for in vivo experiments, the cells were infected with the GFP viral particles (sc-108084) or Orange viral particles (pWPXL-Orange, gift from Dr. Danwei Huangfu). Fluorescence-activated cell sorting was used to remove unlabeled cells. To establish NDST1 knockdown clonal lines, single cells were seeded into a 96-well plate at one cell per well. NDST1 knockdown cell lines (shGFP-14, shGFP-17, shORG-8, and shORG-10) and knockdown control cells (CtGFP) were established after characterization by RT-qPCR for NDST1 gene expression and immunostaining for heparan sulfate expression. Some untargeted cells were also labeled with only pWPXL-Orange and these cells expressed the same level of NDST1 as the CtGFP cells, therefore these cells were also used as controls (CtORG) in some experiments (FIG. 1A). Reverse transcription was conducted using High Capacity cDNA Reverse Transcription Kit (Life Technologies), while RNA extraction, purification, and RT-qPCR were performed following previously established protocols utilizing NDST1 primers purchased from Santa Cruz. To evaluate cell proliferation rates, 40K cells were seeded into each well of a 12-well plate and at 24 hour time intervals until day 5; cells were collected and counted using the Vi-CELL Series Cell Viability Analyzers (Beckman Coulter).

Heparan Sulfate Immunofluorescence Staining

The NDST1 knockdown clonal lines were grown in monolayers on fibronectin coated slides for two days and were stained for HS following previously established protocols. For comparison, NDST1 knockdown lines were suspended in 3D collagen I gels and stained with the HepSS-1 primary antibody (US Biological) at a 1:100 dilution in 2% goat serum. Both monolayer and 3D samples were incubated with goat anti-mouse IgG secondary antibodies at a 1:300 dilution in 2% goat serum for 1 hour with either Alexa Fluor 555 for the GFP cell lines or Alexa Fluor 488 for the ORG cell lines. The slides were mounted with Vectashield mounting medium containing DAPI (Vector Laboratories), and confocal fluorescent microscopy (Zeiss LSM 510) was used to image cells and quantify HS intensity. ImageJ was used to uniformly subtract background fluorescence based on thresholds measured in negative controls for the HS secondary antibodies. Mean HS fluorescence intensity was determined for all images acquired from staining the 2D monolayers. For the 3D cell suspensions, DAPI and GFP/ORG cell profiles were used to outline individual cells in ImageJ to determine the mean intensity of HS per cell.

Modified-Boyden Chamber for Assessing 3D Matrix Invasion

A modified Boyden chamber was used to apply interstitial flow forces to cells in a three-dimensional culture, mimicking those experienced by tumor cells within the interstitium. See Integr Biol (Camb) 2013; 5(11): 1334-43 for details. Briefly, cells were suspended in type I collagen (4 mg/ml), exposed to fluid flow forces via a Darcy flow apparatus (4 h), permitted to migrate for 24 h (without flow) through Transwell chambers (with 8μm pore filters) towards 1 nM TGF-α. Invasion rates were quantified by counting cells present on the underside of the filters. For these tests, only a 4 hour exposure to flow at the lowest shear stress level was applied using a 1 cm H₂O hydrostatic pressure differential to drive the flow. Shear stress transmitted to the cell surface through the glycocalyx layer was estimated to be approximately 0.84 dynes/cm² and the interstitial flow velocity was 0.83 μm/sec as measured previously, which is in the physiologic range of tumor interstitial flows (0.1-2.0 μm/sec). In addition, baseline migration rates (without flow) for all clonal lines were determined. Based on proliferation rates, HS expression, baseline and flow-mediated migration rates, the CtGFP and shORG-8 cell lines were selected as a paired match for continued experimentation.

Microfluidic Device for Quantifying 3D Invasion

The microfluidic device consists of two layers of poly(dimethylsiloxane) PDMS (Sylgard 184, Dow Corning) with the top layer containing the monolithic channel features (50 μm in height) fabricated using soft lithography. The PDMS forms three parallel channels along the device, each with independent inlet and outlet ports. A mixture of Type 1 collagen gel (3 mg/ml) and fibronectin (10 μg/ml) (both from BD Biosciences) was aspirated into the middle channel and allowed to polymerize. A concentrated solution (˜10⁶ cells/ml) of either CtGFP or shORG-8 cells was then introduced along the length of the top channel. Along the length of the middle channel, there are seven apertures, each 50 μm high and 100 μm wide, where the cancer cells from the top channel can contact the matrix. After the tumor cells were confluent, interstitial flow of medium was introduced via a pressure drop across the collagen gel in the direction perpendicular to the cells covering the apertures. Interstitial flow was maintained for 12 hours with a programmable syringe pump (Harvard Apparatus), and the flow velocity was estimated to be 9.5 μm/s based on the previously reported inlet flow rate and channel geometry. Following interstitial flow priming, media containing 1 nM TGF-α was applied to the bottom microchannel to impart a chemoattractant gradient across the collagen gel. The interstitial flow primed conditions were compared to static (no flow) controls where cells were maintained for 12 hours with static fluid reservoirs followed by 12 hours of an applied TGF-α chemoattractant gradient. Fluorescence images were acquired at T=0, 12, and 24 hours after the start of each experiment with an epi-fluorescence microscope (Olympus IX70, OpenLab software) for both the Flow Primed and Static control conditions.

Animal Models for Single Clone and Combined Clone Tumors

Both CtGFP (control) and shORG-8 (NDST1 knockdown) tumor cells were maintained in Eagle's MEM (Life Technologies) supplemented with 10% fetal bovine serum. Cells from sub-confluent monolayers were harvested by trypsinization and resuspended in MEM to a final concentration of 2×10⁶ cells/ml. Male severe combined immunodeficient mice (SCID: C.B-17/Icr-SCID/Sed), 6-10 weeks of age, bred in the gnotobiotic animal facility of the Steele Laboratories, MGH, were used in accordance with a protocol approved by the Massachusetts General Hospital. Mice were anesthetized using a ketamine (100 mg/kg body weight; Parke-Davis, Morris Plains, N.J.) and xylazine (10 mg/kg body weight; Miles, Shawnee Mission, Kans.) mixture administered by i.m. injection. For tumor implantation, the hair on the left flank was first shaved; a small left lateral laparotomy in the kidney area was performed, and the kidney was carefully exteriorized. For the single clone model, a suspension of 1×10⁶ cells (CtGFP or shORG-8 clonally expanded from single cells) in 0.1 ml of MEM was slowly injected under the capsule of the kidney; for the combined clones model, 1×10⁶ cells containing an equal mixture of both clonally expanded cell lines was injected. The retroperitoneal wall was sutured with 5-0 prolene (Ethicon, Somerville, N.J.) and the skin closed with wound clips. To allow secondary colonies time to develop, the primary tumors were removed via nephrectomy four weeks after implantation. When tumor burden from the metastases was evident, the mice were sacrificed and the lungs, liver, spleen, and contralateral kidney along with abdominal and retroperitoneal structures were harvested and fixed in 4% paraformaldehyde.

Tissue Processing and Analysis

The harvested tissue was embedded in OCT medium and sectioned to the center of the tissue; 10 μm (thin) sections were then collected for H&E staining and adjacent 200 μm (thick) sections were collected for fluorescence microscopy. The H&E sections were imaged using an Olympus microscope with a Canon SLR while the thick sections were analyzed using a Nikon stereomicroscope equipped for fluorescence detection and fitted with a Nikon D70 SLR. In both cases, images of the entire tissue were collected for analysis. For fluorescence imaging, the CtGFP containing samples were imaged using the FITC filter set and the shORG-8 cells were imaged with the rhodamine filter set. Each H&E section of tissue was analyzed under light microscopy to identify the native tissue and each sample was categorized by tissue type. Both H&E and fluorescence imaging were analyzed together to identify tumor cells and tumor colonies throughout each sample, then histological analysis was performed to characterize the morphology and phenotype of tumor cells and tumor-native tissue interaction. Tumor colonies were categorized as being either non-invasive (masses growing separate from the organ system that did not invade the capsule) or invasive (cells that crossed the tumor-normal tissue boundary to infiltrate the parenchyma to create an undefined border). Metastases in distant organs and invasive tumor masses near the primary site were categorized together. For the combined/mixed-cells model, tumor colonies were first identified as either originating from CtGFP or shORG cells by fluorescence imaging and were analyzed separately.

Statistical Analysis

All quantitative data were normalized by their respective controls (NDST1 to GAPDH, cell growth rates to cell numbers at T=0, cell migration to respective no-flow cases, and HS fluorescence to CtGFP or CtORG cells) and are presented as mean±standard error of the mean. The two-tailed Student's t-test was utilized on the quantitative normalized data and comparisons were made to respective no-flow control cell lines to determine statistical significance in all these cases. Regression analysis was performed on cell growth rates utilizing Student's t-test methods for comparing multiple regression coefficients and elevations. Chi-square with Yates correction analysis was performed for comparison of all animal categorical data within the control and knockdown cases and for quantifying changes in invasion and metastatic rates for each type of tissue. Further analysis by the McNemar test was utilized for the paired categorical data in the combined clones model. Significant values were set at p<0.05 with Bonferroni corrections applied for multiple comparisons where required.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method for inhibiting metastasis of cancer cells, method comprising steps of:
treating an in vivo tumor with a heparan sulfate (HS) inhibitor wherein the heparan sulfate (HS) inhibitor causes a decrease in glycosaminoglycan concentration on a surface of cells in the in vivo tumor relative to untreated cells.

2. The method as recited in claim 1, wherein the heparan sulfate (HS) inhibitor is a shRNA transfected to inactivate N-deacetylase/N-sulfotransferase1 (NDST1) gene.

3. The method as recited in claim 1, wherein the heparan sulfate (HS) inhibitor is CRISPR-Cas9 transfected to inactivate NDST1 gene, thus genetically modifying the in vivo tumor to decrease heparan sulfate (HS) production.

4. (canceled)

5. (canceled)

6. The method as recited in claim 1, wherein the heparan sulfate (HS) inhibitor is heparinase III, the heparinase III cleaving heparan sulfate (HS) on the surface of the cells in the in vivo tumor.

7. (canceled)

8. The method as recited in claim 1, wherein the heparan sulfate (HS) inhibitor is a MAP kinase inhibitor.

9. The method as recited in claim 1, wherein the heparan sulfate (HS) inhibitor is selected from the group consisting of GNE-470, trametinib and fucoxanthin.

10. The method as recited in claim 1, wherein the in vivo tumor is an in vivo renal carcinoma tumor, and the heparan sulfate (HS) inhibitor is selected from a group consisting of:
(a) shRNA transfected to inactivate N-deacetylase/N-sulfotransferase1 (NDST1) gene;
(b) CRISPR-Cas9 transfected to inactivate NDST1 gene;
(c) Heparinase III;
(d) MAP kinase inhibitor;
(e) from the specific group consisting of GNE-470, trametinib and fucoxanthin.

11. A method for inhibiting metastasis of cancer cells, the method comprising sequential steps of:
surgically removing at least a portion of a tumor from an in vivo surgical location of a patient;
treating the in vivo surgical location with heparan sulfate (HS) inhibitor wherein the heparan sulfate (HS) inhibitor causes a decrease in glycosaminoglycan concentration on a surface of cells in the in vivo surgical location relative to untreated cells, thereby inhibiting metastasis of any residual cancer cells in the in vivo surgical location.

12. The method as recited in claim 11, wherein the in vivo tumor is an in vivo renal carcinoma tumor.

13. The method as recited in claim 11, wherein the heparan sulfate (HS) inhibitor is a shRNA transfected to inactivate N-deacetylase/N-sulfotransferase1 (NDST1) gene.

14. The method as recited in claim 11, wherein the heparan sulfate (HS) inhibitor is CRISPR-Cas1 transfected to inactivate NDST1 gene, thus genetically modifying the in vivo tumor to decrease heparan sulfate (HS) production.

15. (canceled)

16. (canceled)

17. The method as recited in claim 11, wherein the heparan sulfate (HS) inhibitor is heparinase III, the heparinase III cleaving heparan sulfate (HS) on the surface of the cells in the in vivo tumor.

18. (canceled)

19. The method as recited in claim 11, wherein the heparan sulfate (HS) inhibitor is selected from the group consisting of GNE-470, trametinib and fucoxanthin.

20. (canceled)

21. The method as recited in claim 11, wherein the heparan sulfate (HS) inhibitor is a MAP kinase inhibitor.

22. The method as recited in claim 11, wherein the tumor is a renal carcinoma tumor wherein the heparan sulfate (HS) inhibitor is a shRNA transfected into a N-deacetylase/N-sulfotransferase1 (NDST1) gene of the cells in the in vivo surgical location.

23. The method as recited in claim 11, wherein the tumor is a renal carcinoma tumor wherein the heparan sulfate (HS) inhibitor is CRISPR-Cas1 transfected to inactivate NDST1 gene, thus genetically modifying the in vivo tumor to decrease heparan sulfate (HS) production.

24. The method as recited in claim 11, wherein the tumor is a renal carcinoma tumor wherein the heparan sulfate (HS) inhibitor is heparinase III, the heparinase III cleaving heparan sulfate (HS) on the surface of the cells in the in vivo tumor.

25. The method as recited in claim 11, wherein the tumor is a renal carcinoma tumor wherein the heparan sulfate (HS) inhibitor is selected from the group consisting of GNE-470, trametinib and fucoxanthin.

26. A method for inhibiting metastasis of cancer cells, method comprising steps of:
treating an in vivo tumor with a hyaluronic acid (HA) inhibitor wherein the hyaluronic acid (HA) inhibitor causes a decrease in glycosaminoglycan concentration on a surface of cells in the in vivo tumor relative to untreated cells;
wherein the hyaluronic acid (HA) inhibitor is selected from a group consisting of:
(a) a shRNA transfected into inactivate Hyaluronan synthase 1 (HAS1) gene, thus genetically modifying the in vivo tumor to decrease hyaluronic acid (HA) production;
(b) a CRISPR-Cas9 transfected to inactivate HAS1 gene, thus genetically modifying the in vivo tumor to decrease hyaluronic acid (HA) production; and
(c) a hyaluronidase, the hyaluronidase cleaving hyaluronic acid (HA) on the surface of the cells in the in vivo tumor.
